(19) United States
(12) Patent Application Publication
(10) Pub. No.: US 2024/0374428 A1
(43) Pub. Date: Nov. 14, 2024

(54) MONITORING LASER-TISSUE INTERACTION DURING FEMTOSECOND LASER INCISION IN CORNEA USING BACK-REFLECTED TREATMENT LIGHT

(71) Applicant: AMO Development, LLC, Irvine, CA (US)

(72) Inventors: Alireza Malek Tabrizi, Fremont, CA (US); Griffith Altmann, Ladera Ranch, CA (US); Harvey Liu, Fremont, CA (US); Zenon Witowski, Pleasanton, CA (US); Mohammad Saidur Rahaman, San Jose, CA (US); Hong Fu, Pleasanton, CA (US)

(21) Appl. No.: 18/661,507

(22) Filed: May 10, 2024

Related U.S. Application Data
(60) Provisional application No. 63/501,537, filed on May 11, 2023.

(51) Int. Cl. A61F 9/008

(52) U.S. Cl. CPC A61F 9/00827; A61F 9/0084; A61F 2009/00844; A61F 2009/00872

(57) ABSTRACT

During laser ophthalmic procedures, back-reflected treatment laser light is detected by an auto-Z module and analyzed in real-time to determine various aspects of laser-tissue interaction during the procedure. This method can detect the presence of “black spots” (locations where no laser-tissue interaction occurred), sub-optimal incision quality, etc. in real time, and allows for dynamical adjustment of the laser treatment parameters such as pulse energy, laser spot separation, etc. to correct the detected problems. The auto-Z signal analysis may also depend on which incision segment or region is currently being cut, to optimally control different cutting segments. This method improves corneal incision quality and helps to achieves consistent laser-tissue interaction from patient to patient.

### CROSS REFERENCE TO RELATED APPLICATIONS

This application claims priority to U.S. Provisional Patent Application No. 63/501,537, filed on May 11, 2023, the entire contents of which are hereby incorporated by reference.

### BACKGROUND OF THE INVENTION

This invention relates to ophthalmic laser surgery systems and methods, and in particular, it relates to determination of laser-tissue interaction and cut quality using back-reflected surgical beam during femtosecond laser incisions in cornea.

Femtosecond lasers are used to cut different types of corneal incisions such as flaps, lenticule incisions, keratoplasty incisions, etc. The gas bubbles generated by laser pulses in the cornea sometimes results in tissue movement and blocking of laser beam which may create uncut islands or affect the quality of subsequent cut segments. It is critical to optimize the laser cut parameters such as pulse energy and laser focal spot separation to avoid generation of excessive bubbles.

The optimization of laser incision parameters may be done by cutting ex-vivo or in-vivo eyes, and examining the video images of the eye during cutting and dissecting the cut incision. Observation of large bubbles in the cutting video and presence of tissue bridges or tissue roughness in the location the observed bubbles may be an indication for the need to reduce pulse energy or increase laser spot separation.

### SUMMARY OF THE INVENTION

The present invention is directed to ophthalmic laser surgery systems and methods that substantially obviates one or more of the problems due to limitations and disadvantages of the related art.

An object of the present invention is to monitor back-reflected treatment beam to detect effects of laser-tissue interaction and improve incision quality.

Additional features and advantages of the invention will be set forth in the descriptions that follow and in part will be apparent from the description, or may be learned by practice of the invention. The objectives and other advantages of the invention will be realized and attained by the structure particularly pointed out in the written description and claims thereof as well as the appended drawings.

To achieve the above objects, the present invention provides a method implemented in an ophthalmic laser system, which includes: (a) delivering a treatment laser beam to an eye tissue based on a plurality of laser treatment parameters to form incisions in the eye tissue; (b) continuously measuring an intensity value of a portion of a back-reflected treatment beam from the eye tissue; (c) continuously comparing the intensity value of the back-reflected treatment beam to a threshold intensity value in real time; (d) when the intensity value of the back-reflected treatment beam is below the threshold intensity value, adjusting at least some of the laser treatment parameters in real time; and (e) delivering the treatment laser beam to the eye tissue based on the adjusted laser treatment parameters to form the incisions in the eye tissue.

The above method may further include: obtaining position data representing a location of the laser beam delivered in the eye; based on the position data, identifying a region of the incisions that is currently being formed; and selecting the threshold intensity value corresponding to the identified region of the incisions that is currently being formed.

In another aspect, the present invention provides a method implemented in an ophthalmic laser system, which includes: (a) delivering a treatment laser beam to an eye tissue based on a plurality of laser treatment parameters to form incisions in the eye tissue; (b) continuously measuring an intensity value of a portion of a back-reflected treatment beam from the eye tissue; (c) continuously comparing the intensity value of the back-reflected treatment beam to previous intensity values; (d) when the intensity value drops by more than a threshold amount within a predetermined time interval, adjusting at least some of the laser treatment parameters in real time; and (e) delivering the treatment laser beam to the eye tissue based on the adjusted laser treatment parameters to form the incisions in the eye tissue.

The above method may further include: obtaining position data representing a location of the laser beam delivered in the eye; based on the position data, identifying a region of the incisions that is currently being formed; and selecting the threshold amount based on the identified region of the incisions that is currently being formed.

In yet another aspect, the present invention provides a method implemented in an ophthalmic laser system, which includes: (a) delivering a treatment laser beam to an eye tissue based on a plurality of laser treatment parameters to form incisions in the eye tissue; (b) continuously measuring an intensity value of a portion of a back-reflected treatment beam from the eye tissue; (c) continuously analyzing the intensity value of the back-reflected treatment beam based on predefined statistical characteristics to determine incision quality; (d) when the incision quality is determined to be sub-optimal, adjusting at least some of the laser treatment parameters in real time; and (e) delivering the treatment laser beam to the eye tissue based on the adjusted laser treatment parameters to form the incisions in the eye tissue.

The above method may further include: obtaining position data representing a location of the laser beam delivered in the eye; based on the position data, identifying a region of the incisions that is currently being formed; and selecting the statistical characteristics based on the identified region of the incisions that is currently being formed.

In yet another aspect, the present invention provides a method implemented in an ophthalmic laser system, which includes: (a) delivering a treatment laser beam to an eye tissue based on a plurality of laser treatment parameters to form incisions in the eye tissue; (b) continuously measuring an intensity value of a portion of a back-reflected treatment beam from the eye tissue; (c) obtaining position data representing a location of the laser beam delivered in the eye, and based on the position data, identifying regions of the incisions that are currently being formed; for each of at least two different regions of the incision, (d) selecting analysis criteria based on the identified region of the incision, wherein the analysis criteria for the two different regions are different; (e) continuously analyzing the intensity value of the back-reflected treatment beam based on selected criteria; (f) adjusting at least some of the laser treatment parameters in real time based on results of the analysis in step (e); and (g) delivering the treatment laser beam to the eye tissue based on the adjusted laser treatment parameters to form the incisions in the region.

It is to be understood that both the foregoing general description and the following detailed description are exemplary and explanatory and are intended to provide further explanation of the invention as claimed.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 schematically illustrates a method of monitoring real-time auto-Z signal to determine aspects of laser-tissue interaction according to embodiments of the present invention.

FIG. 2 show three examples of auto-Z signals.

FIG. 3 schematically illustrates a method of monitoring real-time auto-Z signal to detect black spots according to an embodiment of the present invention.

FIG. 4 schematically illustrates a method of monitoring real-time auto-Z signal to detect black spots according to an alternative embodiment of the present invention.

FIG. 5 schematically illustrates a method of monitoring real-time auto-Z signal to achieve consistent laser-tissue interaction from patient to patient according to an embodiment of the present invention.

FIG. 6 schematically illustrates an alternative method of monitoring real-time auto-Z signal to determine aspects of laser-tissue interaction according to additional embodiments of the present invention.

FIG. 7 and FIG. 8 schematically illustrate an ophthalmic surgical laser system in which a bubble detection method according to embodiments of the present invention may be implemented.

FIG. 9 schematically illustrates an auto-Z module in the laser system of FIGS. 7 and 8.

FIG. 10 schematically illustrates another auto-Z module in the laser system of FIGS. 7 and 8.

### DETAILED DESCRIPTION OF THE INVENTION

Embodiments of the present invention provide methods of optimization, testing and control of femtosecond laser treatment parameters such as pulse energy, fast blanking parameters (which controls pulse frequency applied to the eye tissue), laser focal spot separation, etc. More specifically, embodiments of the present invention use real-time measurement of back-reflected light signal from the eye under treatment (referred to as auto-Z signal) to monitor laser-tissue interaction during cutting, to detect occurrences of various abnormal laser-tissue interactions, and to make real-time adjustments of treatment parameters in response.

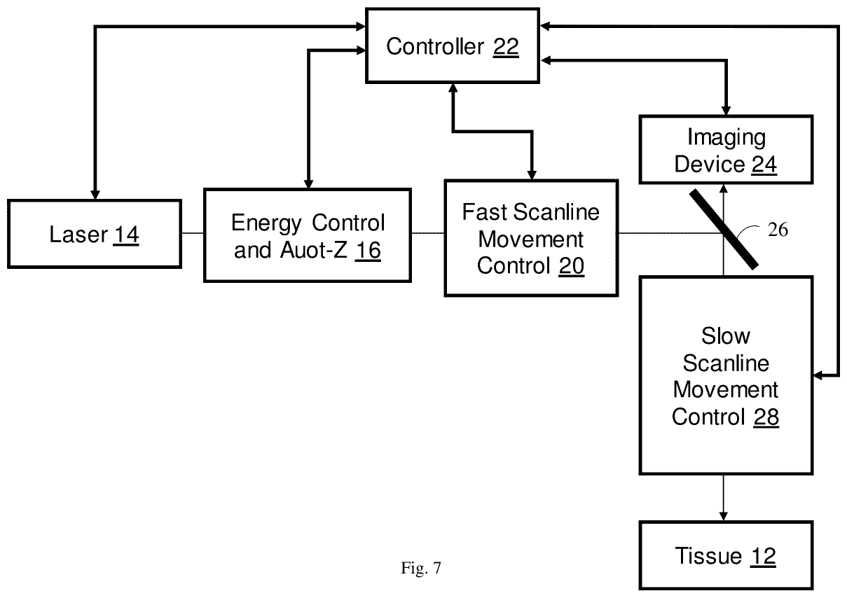

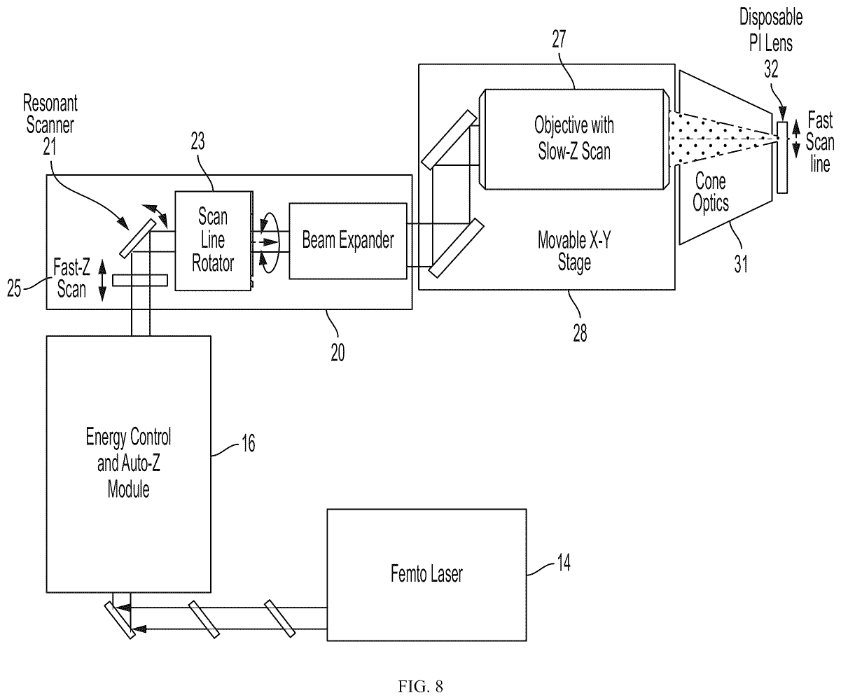

FIGS. 7 and 8 schematically illustrate an ophthalmic surgical laser system in which an auto-Z signal monitoring method according to embodiments of the present invention may be implemented. FIG. 7 shows a laser system for making incisions (cuts) in a tissue 12 of a patient's eye. The system includes, but is not limited to, a laser 14 capable of generating a pulsed laser beam, an energy control module 16 for varying the pulse energy of the pulsed laser beam, a fast scanline movement control module 20 for generating a fast scanline of the pulsed laser beam (described in more detail later), a controller 22, and a slow scanline movement control module 28 for moving the laser scanline and delivering it to the tissue 12. The controller 22, such as a processor operating suitable control software, is operatively coupled with the fast scanline movement control module 20, the slow scanline movement control module 28, and the energy control module 16 to direct the scanline of the pulsed laser beam along a scan pattern on or in the tissue 12. In this embodiment, the laser system further includes a beam splitter 26 and a imaging device 24 coupled to the controller 22 for a feedback control mechanism (not shown) of the pulsed laser beam. Other feedback methods may also be used. In an embodiment, the pattern of pulses may be summarized in machine readable data of tangible storage media in the form of a treatment table. The treatment table may be adjusted according to feedback input into the controller 22 from an automated image analysis system in response to feedback data provided from a monitoring system feedback system (not shown).

Laser 14 may comprise a femtosecond laser capable of providing pulsed laser beams, which may be used in optical procedures, such as localized photodisruption (e.g., laser induced optical breakdown). Localized photodisruptions can be placed at or below the surface of the tissue or other material to produce high-precision material processing. For example, a micro-optics scanning system may be used to scan the pulsed laser beam to produce an incision in the material, create a flap of the material, create a pocket within the material, form removable structures of the material, and the like. The term “scan” or “scanning” refers to the movement of the focal point of the pulsed laser beam along a desired path or in a desired pattern.

FIG. 8 is another exemplary diagram of the laser system. FIG. 8 shows components of a laser delivery system including a moveable XY-scanner (or movable XY-stage) 28 of a miniaturized femtosecond laser system. In this embodiment, the system uses a femtosecond oscillator, or a fiber oscillator-based low energy laser. This allows the laser to be made much smaller. The laser-tissue interaction is in the low-density-plasma mode. An exemplary set of laser parameters for such lasers include pulse energy in the 40-100 nJ range and pulse repetitive rates (or “rep rates”) in the 2-40 MHz range. A fast-Z scanner 25 and a resonant scanner 21 direct the laser beam to a scanline rotator 23. When used in an ophthalmic procedure, the laser system also includes a patient interface device that has a fixed cone nose 31 and a contact lens (disposable patient interface lens) 32 that engages with the patient's eye. A beam splitter may be placed inside the cone 31 of the patient interface to allow the whole eye to be imaged via visualization optics. In some embodiments, the laser system may use: optics with a 0.6 numerical aperture (NA) which would produce 1.1 μm Full Width at Half Maximum (FWHM) focus spot size; and a resonant scanner 21 that produces 0.2-1.2 mm scan line with the XY-scanner scanning the resonant scan line to a 1.0 mm field. The prism 23 (e.g., a Dove or Pechan prism, or the like) rotates the resonant scan line in any direction on the XY plane. The fast-Z scanner 25 sets the incision depth. The slow scanline movement control module employs a movable XY-stage 28 carrying an objective lens with Z-scanning capability 27, referred to as slow-Z scanner because it is slower than the fast-Z scanner 25. The movable XY-stage 28 moves the objective lens to achieve scanning of the laser scanline in the X and Y directions. The objective lens changes the depth of the laser scanline in the tissue. The energy control and auto-Z module 16 may include appropriate components to control the laser pulse energy, including attenuators, etc. It also includes an auto-Z module which employs a confocal or non-confocal imaging system to provide a depth reference. The miniaturized femtosecond laser system may be a desktop system so that the patient sits upright while being under treatment. This eliminates the need of certain opto-mechanical arm mechanism(s), and greatly reduces the complexity, size, and weight of the laser system. Alternatively, the miniaturized laser system may be designed as a conventional femtosecond laser system, where the patient is treated while lying down.

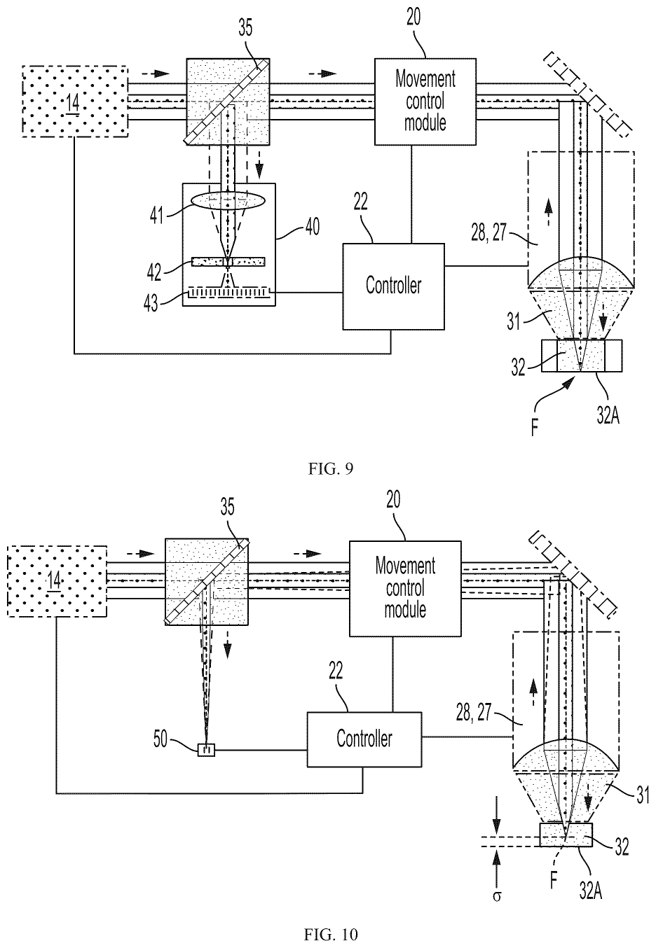

Examples of the auto-Z module is described in more detail with reference to FIGS. 9 and 10. FIG. 9 illustrates an auto-Z module that employs a confocal optical system. As shown in FIG. 9, a part of the laser beam generated by the laser source 14 passes through a beam splitter 35, and after passing through other optical components including the scanning devices (e.g. movement control module 20), is focused by the objective lens 27. The laser light that exits the objective lens is partially reflected by the eye tissue and/or other optical elements such as the lower surface 32A of the contact lens 32, and the reflected laser light travels backwards into the objective lens. Note that in the example of FIG. 9, the laser beam focus F is shown as being located at the contact lens lower surface 32A, but this is only an example. After the back-reflected laser light is focused by the objective lens 27 into a parallel beam and pass through the other optical components including the scanning devices, a part of the reflected laser light is reflected by the beam splitter 35 into the confocal detection assembly 40.

The confocal detection assembly 40 includes a lens 41 (referred to as the confocal lens), a pinhole 42, and a light intensity detector 43 such as photodiodes. The confocal lens 41 is configured to focuses the parallel laser beam to the pinhole 42, and the light that passes through the pinhole is detected by the detector 43. Due to the presence of the pinhole, only light reflected by the volume of sample (e.g. eye tissue) located at the focal point of the laser beam will pass through the pinhole and contribute significantly to the detected confocal signal (auto-Z signal).

FIG. 10 illustrates an auto-Z module that employs a non-confocal optical system. As shown in FIG. 10, a part of the laser beam generated by the laser source 14 passes through a beam splitter 35, and after passing through other optical components including the scanning devices (e.g. movement control module 20), is focused by the objective lens 27. In a preferred embodiment, the objective lens 27 has a relatively high numerical aperture (NA), for example, approximately 0.4 or higher. The focus spot size produced by the objective lens is preferably as small as 2 μm, or 1 μm, or even smaller. The laser light that exits the objective lens 27 is partially reflected by the eye tissue and/or other optical elements such as the lower surface 32A of the contact lens 32, and the reflected laser light travels backwards into the objective lens. After the back-reflected light is focused by the objective lens 27 and passing through other optical components, a part of the reflected light is reflected by the beam splitter 35 onto a small two-dimensional light intensity detector 50 (e.g. an image sensor). No confocal lens or pinhole is used in front of the detector 50.

Because of the absence of the confocal lens, the back-reflected light may be focused or defocused at the detector 50, depending on the depth at which the light is reflected. The optical system parameters (e.g., the optical power of the objective lens and the distances between various optical components) are designed such that when a reflective surface is located at a predefined distance δ below the focal point F of the laser beam, the light reflected from that surface will be focused onto the detector 50 and generate a relatively strong auto-Z signal on the detector. Light reflected from structures at other distances will be de-focused at the plane of the detector 50 and generate a relatively weak auto-Z signal due to the small area of the detector 50. In the example shown in FIG. 10, the laser beam is shown as being focused to a focal point F located at a distance δ above the lower surface 32A of the contact lens 32, and the light reflected by this surface is focused onto the non-confocal detector 50. The optical principles of the non-confocal detection system is described in more detail in commonly owned U.S. Pat. Appl. Pub. No 20200064622, entitled Detection Of Optical Surface Of Patient Interface For Ophthalmic Laser Applications Using A Non-Confocal Configuration.

Embodiments of the present invention utilize the real-time auto-Z signal, which is in the form of light intensity as a function of time, to detect and monitor various aspects of the laser-tissue interaction during laser ophthalmic procedures. As illustrated in FIG. 1, during laser treatment (step S101), back reflected treatment laser beam is directed into the auto-Z detector 40 or 50 (step S102), and the detected signal is digitized and sampled, for example at 1 KHz frequency, to generate an auto-Z signal (step S103). The auto-Z signal is analyzed (e.g., by the controller) to determine various aspects of the laser-tissue interaction during the treatment procedure (step S104). The aspects of the laser-tissue interaction includes, for example, presence of “black spots”, patient-to-patient variations, location-dependent monitoring, etc., as will be described in more detail later.

The inventors of the present invention has demonstrated that the intensity of the auto-Z signal is responsive to laser treatment parameters such as pulse energy. In one example, the intensity of the auto-Z signal was plotted against the x, y and z coordinates of the laser spot position. The auto-Z vs. x, y, z coordinates plot revealed the locations and relative sizes of bubbles in femtosecond laser incision, as bubble or gap in the surgical beam path tent to result in larger auto-Z signals. Experiments of corneal flap cuts and corneal lenticule cuts conducted by the inventors showed that the relative intensity of the auto-Z signal depended on the pulse energies.

Other laser treatment parameters such as spot separation (including line to line spacing of the scanlines) and laser blanking may also impact the bubble generation in tissue and therefore the auto-Z signal strength. Thus, analyses of the auto-Z signals may be used in control, test and optimization of laser spot separation and pulse energy in corneal tissue dissection. It can also be used for determination of any gap in the laser beam path.

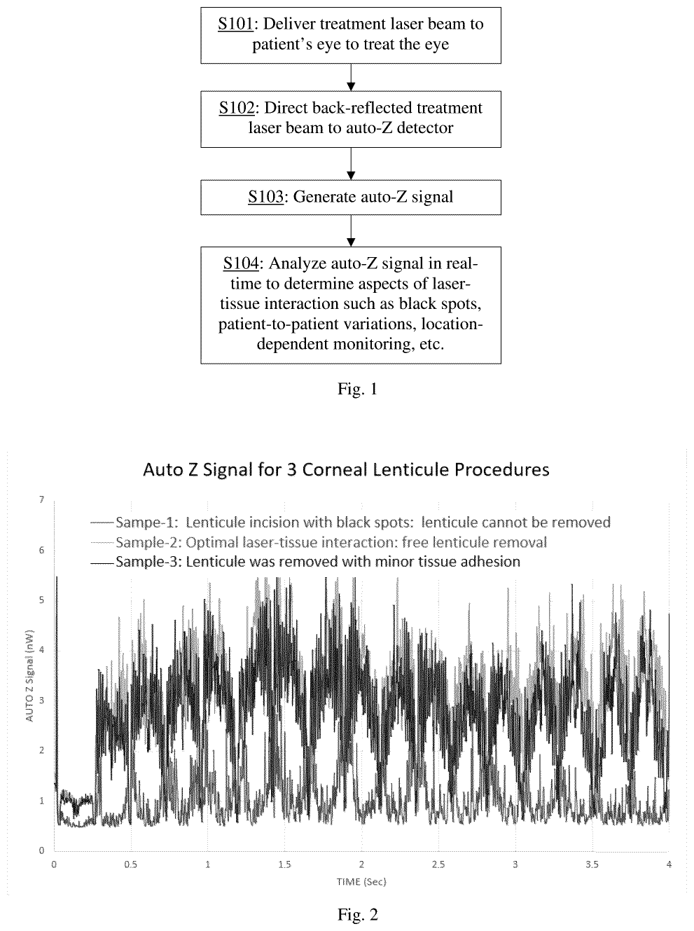

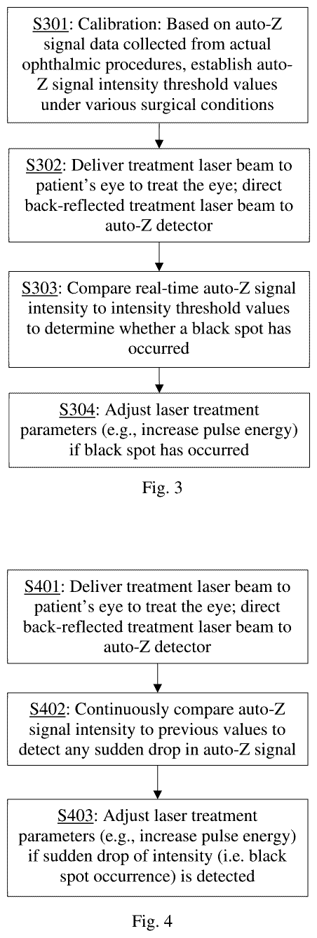

In one embodiment (FIG. 3), the auto-Z signal monitoring is used to address the “black spots” problem. In femtosecond laser surgery, “black spots” refer to locations in the tissue where there is no laser-tissue interaction, so the tissue is not cut. Black spots are a rare but a serious procedure complication. When many black spots happen, the incision cannot be well formed. In a corneal lenticule extraction procedure, for example, this results in the inability to remove the lenticule, and a follow up retreatment is required. It is worth noting that even for the same laser setting, the tissue response to laser is different from patient to patient, so black spots can occur from time to time. FIG. 2 shows three exemplary auto-Z signals measured during three corneal lenticule incision procedures. In FIG. 2, Sample-1 is an example of the auto-Z signal for a lenticule incision procedure where black spots occurred, while in Sample-2 and Sample-3, the incisions were normal without black spots. It can be seen that the auto-Z signal in Sample-1 is significantly lower than the normal examples because there was less plasma-induced cavitation bubble and light scattering from these bubbles. Using the auto-Z signal, the occurrence of a black spot may be dynamically detected (i.e. while treatment is ongoing), and the treatment process may be dynamically adjusted, for example, by raising the laser energy to above the normal cutting threshold for this specific corneal tissue where black spots occurred.

Thus, a method according to one embodiment of the present invention, shown in FIG. 3, includes comparing the intensity (absolute values) of the real-time auto-Z signal to an auto-Z signal intensity threshold to determine whether a black spot has occurred (step S303). As a calibration, the auto-Z signal intensity threshold is established empirically beforehand, based on auto-Z signal data collected from actual ophthalmic procedures on multiple eyes under various surgical conditions (laser pulse energy, etc.) where black spots occurred for some and did not occur for others (step S301). The calibration establishes the normal auto-z signal intensity ranges and the threshold for each of multiple sets of surgical conditions (treatment parameters). While performing laser treatment on a patient's eye, the auto-Z signal is monitored (step S302), and based on the comparison of the auto-Z signal intensity to the auto-Z signal intensity threshold under the same surgical condition, the controller determines whether a black spot has occurred (step S303), and adjusts the laser treatment parameters accordingly (step S304). Both the monitoring (step S303) and adjustment (step S304) are performed automatically by the controller. For example, the controller may automatically increase the laser pulse energy by 5-10 nJ when the observed auto-z signal is less than a threshold which is 30% of the average intensity value established in the calibration.

As an alternative, because black spots typically occurs only during parts of the same procedure, a sudden drop of the auto-z signal during cutting may also be used as an indication of a black spot occurrence. Thus, as shown in FIG. 4, a method according to an alternative embodiment of the present invention includes continuously monitoring the auto-Z signal during treatment (step S401); when a sudden drop in the auto-Z signal intensity is detected (step S402), which indicates the occurrence of a black spot, the controller automatically adjusts the laser treatment parameters (step S403). The criteria for detecting a sudden drop may be set suitably; for example, a drop of more than 30% within the time interval of a few laser pulses or a few scanlines may be deemed a sudden drop in intensity. No calibration is required for this alternative method.

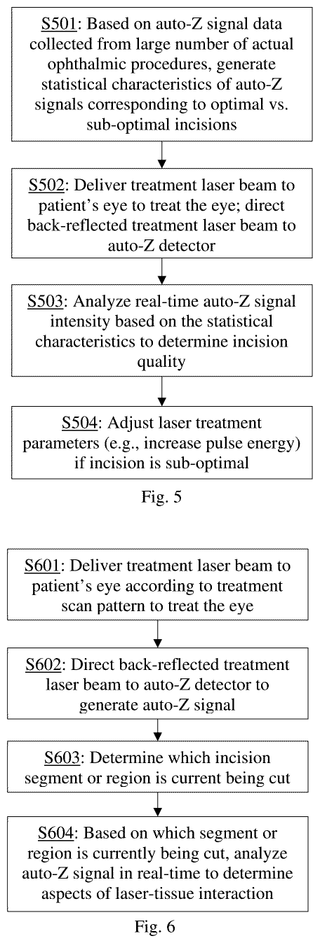

In another embodiment (FIG. 5), the auto-Z signal is used to achieve consistent laser-tissue interaction from patient to patient. Typically, the cornea response to laser treatment is different from patient to patient; as a result, the optimum laser treatment parameters may be different for different patients. For example, the same laser pulse energy may be too low for some patients and causes tissue adhesion (insufficient cutting), or too high for some other patients and causes tissue roughness. Monitoring the auto-Z signal during treatment and adjusting the laser treatment parameters accordingly can help achieve more consistent incisions from patient to patient. In the examples shown in FIG. 2, Sample-3 was obtained for a normal lenticule incision with minor tissue adhesion, and Sample-2 was obtained for a normal tissue-bridge-free lenticule incision where the lenticule could be removed without any manual dissection. The analyses of the auto-Z signal may use different signal signatures, such as average strength, and/or standard deviation of the signal, to judge cutting average level and cutting interface smoothness.

More specifically, as shown in FIG. 5, statistical characteristics of the auto-Z signals corresponding to optimal incisions (e.g., those that are tissue-bridge-free) versus sub-optimal incisions (e.g. those that cause minor tissue adhesion) are generated empirically from data gathered in a large number of actual incision procedures (step S501). For example, a matrix of multi-variables (signal mean and standard deviation at different cutting locations) may be established, then common features for the best cuts may be found by statistical analysis. Then, when performing incisions on individual patients, the real-time auto-Z signal is monitored (step S502) and analyzed using the statistical characteristics to judge the cut quality (step S503) and to determine whether laser treatment parameters should be dynamically adjusted during cutting (step S504). For example, if the auto-Z signal intensity deviates from the average of the statistics in either direction by 30% or more, the current cutting is judged to be suboptimal, and the laser pulse energy is adjusted by 1-3 nJ in the corresponding direction (increase or decrease based on whether the signal is smaller or greater than the average).

In yet another embodiment (FIG. 6), the auto-Z signal is used to optimally control different cutting segments. A corneal procedure typically includes cutting multiple segments of the cornea, such as top and bottom lenticule cuts in a lenticule extraction procedure, or the bed cut and side cut in a flap procedure, etc. Each cut segment may have different regions such as center, edge, etc. These different cutting segments or regions may require different control strategies. For example, in the method of detecting black spots (FIG. 3), at the lenticule edge, which is outside the visual field, the threshold values used in step S303 may be different from that used at the lenticule center which is in the patient's visual field.

During treatment, the controller records the laser focus spot position as represented by values of various scanning motor encoders (e.g., those in the XY scanner, fast Z and slow Z scanners), and can determine what segment or region is currently being cut based on the laser focus spot position and the treatment plan being executed (a treatment plan is a specification that describes a pre-set sequence of incisions and incision segments to be formed in the ophthalmic procedure, including their shapes and positions). The criteria used when analyzing the auto-Z signal, e.g. those used in steps S303, S402, and S503, may thus be set to different values for different cutting segments and/or in different regions. Prior to treatment, a calibration process establishes the threshold values and/or other statistical characteristics of the auto-Z signal for difference cutting segments and regions, so that appropriate criteria may be selected in the analyzing steps.

According to this embodiment, as summarized in FIG. 6, during treatment (step S601), while the auto-Z signal is continuously monitored (step S602), the controller determines which incision segment or which region of which segment is currently being cut (step S603). The controller analyzes the auto-Z signal using appropriate control parameters or models based on which incision segment or region is currently being cut, to determine the various aspects of laser-tissue interaction (step S604), such as black spot, etc. as described above.

These location-dependent auto-Z black-spot detection control limits help to minimize false positive, while providing sufficient information to perform dynamic adjustment of laser energy and other laser treatment parameter to reduce the occurrence of black spots.

A dynamic visual display may also be implemented using this technique. For example, a live lenticule drawing may be made during a lenticule procedure. The positions of the cutting point are based on the X, Y, and Z encoder readings, and the color of the point represents the strength of the auto-Z signal. Based on the colors of the live drawing at different locations, the surgeon will be able to know whether this lenticule cutting is normal or abnormal. For example, if the auto-Z signal in cutting posterior lenticule is low, the live graph will show a black posterior surface being drawn. As the controller automatically adjusts the laser pulse energy, the surgeon will see the color of the posterior surface becoming normal again. This visual live graph can also give the surgeon a sense when he/she has done many cases and establish the color of the lenticule on the screen and the extraction and even with later visual outcome and visual recovery.

It will be apparent to those skilled in the art that various modification and variations can be made in the auto-Z signal monitoring and analysis method and related apparatus of the present invention without departing from the spirit or scope of the invention. Thus, it is intended that the present invention cover modifications and variations that come within the scope of the appended claims and their equivalents.

1. A method implemented in an ophthalmic laser system, comprising:
(a) delivering a treatment laser beam to an eye tissue based on a plurality of laser treatment parameters to form incisions in the eye tissue;
(b) continuously measuring an intensity value of a portion of a back-reflected treatment beam from the eye tissue;
(c) continuously comparing the intensity value of the back-reflected treatment beam to a threshold intensity value in real time;
(d) when the intensity value of the back-reflected treatment beam is below the threshold intensity value, adjusting at least some of the laser treatment parameters in real time; and
(e) delivering the treatment laser beam to the eye tissue based on the adjusted laser treatment parameters to form the incisions in the eye tissue.

2. The method of claim 1, further comprising, before step (a):
calculating the threshold intensity value based on calibration data collected from incisions performed on multiple other eyes, wherein some incisions had black spot occurrences and some other incisions had no black spot occurrences.

3. The method of claim 1, wherein the adjusting in step (d) includes increasing a laser pulse energy.

4. The method of claim 1, further comprising:
obtaining position data representing a location of the laser beam delivered in the eye;
based on the position data, identifying a region of the incisions that is currently being formed; and
selecting the threshold intensity value corresponding to the identified region of the incisions that is currently being formed.

5. A method implemented in an ophthalmic laser system, comprising:
(a) delivering a treatment laser beam to an eye tissue based on a plurality of laser treatment parameters to form incisions in the eye tissue;
(b) continuously measuring an intensity value of a portion of a back-reflected treatment beam from the eye tissue;
(c) continuously comparing the intensity value of the back-reflected treatment beam to previous intensity values;
(d) when the intensity value drops by more than a threshold amount within a predetermined time interval, adjusting at least some of the laser treatment parameters in real time; and
(e) delivering the treatment laser beam to the eye tissue based on the adjusted laser treatment parameters to form the incisions in the eye tissue.

6. The method of claim 5, wherein the adjusting in step (d) includes increasing a laser pulse energy.

7. The method of claim 5, further comprising:
obtaining position data representing a location of the laser beam delivered in the eye;
based on the position data, identifying a region of the incisions that is currently being formed; and
selecting the threshold amount based on the identified region of the incisions that is currently being formed.

8. A method implemented in an ophthalmic laser system, comprising:
(a) delivering a treatment laser beam to an eye tissue based on a plurality of laser treatment parameters to form incisions in the eye tissue;
(b) continuously measuring an intensity value of a portion of a back-reflected treatment beam from the eye tissue;
(c) continuously analyzing the intensity value of the back-reflected treatment beam based on predefined statistical characteristics to determine incision quality;
(d) when the incision quality is determined to be sub-optimal, adjusting at least some of the laser treatment parameters in real time; and
(e) delivering the treatment laser beam to the eye tissue based on the adjusted laser treatment parameters to form the incisions in the eye tissue.

9. The method of claim 8, further comprising, before step (a):
calculating the statistical characteristics based on calibration data collected from incisions performed on multiple other eyes, wherein some incisions are tissue-bridge-free and some other incisions have tissue adhesion.

10. The method of claim 8, wherein the adjusting in step (d) includes increasing or decreasing a laser pulse energy.

11. The method of claim 8, further comprising:
obtaining position data representing a location of the laser beam delivered in the eye;
based on the position data, identifying a region of the incisions that is currently being formed; and
selecting the statistical characteristics based on the identified region of the incisions that is currently being formed.

12. The method of claim 8, wherein the statistical characteristics include a matrix of multi-variables including signal mean and standard deviation at different cutting locations.

13. A method implemented in an ophthalmic laser system, comprising:
(a) delivering a treatment laser beam to an eye tissue based on a plurality of laser treatment parameters to form incisions in the eye tissue;
(b) continuously measuring an intensity value of a portion of a back-reflected treatment beam from the eye tissue;
(c) obtaining position data representing a location of the laser beam delivered in the eye, and based on the position data, identifying regions of the incisions that are currently being formed;
for each of at least two different regions of the incision,
(d) selecting analysis criteria based on the identified region of the incision, wherein the analysis criteria for the two different regions are different;
(e) continuously analyzing the intensity value of the back-reflected treatment beam based on selected criteria;
(f) adjusting at least some of the laser treatment parameters in real time based on results of the analysis in step (e); and
(g) delivering the treatment laser beam to the eye tissue based on the adjusted laser treatment parameters to form the incisions in the region.

14. The method of claim 13, wherein the two different regions include a center region and an edge region of a lenticule incision.

15. The method of claim 13, wherein the position data include data from a plurality of scanning motor encoders.
